(19) United States
(12) Patent Application Publication
(10) Pub. No.: US 2006/0206024 A1
(43) Pub. Date: Sep. 14, 2006

(54) Wireless in-bore patient monitor for MRI

(75) Inventors: Arthur R. Weeks JR., Oviedo, FL (US); Stephen Douglas Fisher, Winter Springs, FL (US); Jorgen Kilden-Pedersen, Orlando, FL (US); Robert A. Harwell, Orlando, FL (US); Scott Nolan, Oviedo, FL (US); John C. Moore, Broken Arrow, OK (US)

Correspondence Address: QUARLES & BRADY LLP, 411 E. WISCONSIN AVENUE, SUITE 2040, MILWAUKEE, WI, 53202-4497, US

(73) Assignee: Invivo Corporation

(21) Appl. No.: 11/075,620

(22) Filed: Mar. 9, 2005

(51) Int. Cl. A61B 5/05

(52) U.S. Cl. 600/418

(57) ABSTRACT

A wireless in bore sensor for magnet resonance imaging provides radio frequency communication of physiological and other data signals from a battery powered unit held adjacent to the patient within the bore by using multiple diversity techniques to overcome the interfering environment of the MRI imaging system and to prevent interference with the MRI system.

### CROSS-REFERENCE TO RELATED APPLICATIONS

—

### STATEMENT REGARDING FEDERALLY SPONSORED RESEARCH OR DEVELOPMENT

—

### BACKGROUND OF THE INVENTION

The present invention relates generally to electronic patient monitors, and in particular, to a wireless patient monitor suitable for use in the severe electromagnetic environment of a magnetic resonance imaging machine.

Magnetic resonance imaging (MRI) allows images to be created of soft tissue from faint electrical resonance signals (NMR signals) emitted by nuclei of the tissue. The resonance signals are generated when the tissue is subjected to a strong magnetic field and excited by a radio frequency pulse.

The quality of the MRI image is in part dependent on the quality of the magnetic field, which must be strong and extremely homogenous. Ferromagnetic materials are normally excluded from the MRI environment to prevent unwanted forces of magnetic attraction on these materials and distortion of the homogenous field by these materials.

A patient undergoing an MRI “scan” may be received into a relatively narrow bore, or cavity in the MRI magnet. During this time, the patient may be remotely monitored to determine, for example, heartbeat, respiration, temperature, and blood oxygen. A typical remote monitoring system provides “in-bore” sensors on the patient connected by electrical or optical cables to a monitoring unit outside of the bore.

Long runs of cables can be a problem because they are cumbersome and can interfere with access to the patient and free movement of personnel about the magnet itself.

Desirably, a wireless method of monitoring a patient in the MRI magnet bore would be developed, however, conventional radio transmission faces severe obstacles in the MRI environment. First, the bore of the magnet itself is shielded, restricting the free transmission of radio signals. Second, the frequency and strength of wireless transmissions must be limited to prevent interference with the faint magnetic resonance signals detected by the MRI machine and to accommodate practical battery-powered operation of the transmitter. Third, the radio frequency excitation pulse, that is part of the MRI process, can interfere with wireless transmissions. Finally, the room in which the MRI machine is held may be shielded electrically and magnetically creating problems of reflection of wireless signals such as can produce “dead spots” in the room.

These problems are compounded by the requirement that patient signals, unlike voice signals, for example, must be robust and reliable in real time, even in the face of interference. Particularly, when monitoring signals are used to gate the MRI machine, even short periods of signal dropout or delay are unacceptable. Accordingly, conventional wireless transmission techniques may prove impractical.

### BRIEF SUMMARY OF THE INVENTION

The present invention provides a wireless, in-bore patient monitoring system that provides the necessary high-data transmission rate and robustness against interference in an MRI environment. The invention addressed the difficult environment of MRI by using multiple diversity techniques including frequency diversity, antenna location diversity, antenna polarization diversity and time diversity in the transmitted signals. In the preferred embodiment of the invention, error detection codes attached to the signals or the signal quality of the signals are monitored to select among diverse pathways, dynamically, allowing low error rates and high bandwidth at practical transmission power.

Specifically, the present invention provides a wireless patient sensor system for MRI imaging having a patient unit positionable adjacent to the patient within a bore of an MRI magnet, the patient unit providing at least one sensor receiving a patient signal from the patient and having a wireless transmitter system for transmitting digital data packets communicating the patient signal via wireless signals. A receiving unit having a wireless receiver system receiving the digital data packets from outside the bore of the MRI magnet outputting information of the digital data packets to an operator or as a relay to another device. The wireless transmitter system and wireless receiver system communicate using diverse multiple channels between the patient unit and receiving unit.

It is thus one object of at least one embodiment of the invention to provide a practical wireless communication of patient data from inside a magnet bore in an MRI system.

The receiving unit may compute an error checking code for the digital data packets transmitted on at least two diverse multiple channels to select one diverse multiple channel from which to obtain a digital data packet for outputting. Alternatively the receiving unit may compute a signal quality (e.g., signal strength, time between drop outs, etc.) on the diverse multiple channels to select one diverse multiple channel from which to obtain a digital data packet for outputting.

It is another object of at least one embodiment of the invention to provide a simple and robust method of selecting among the diverse multiple channels to identify accurate data.

The diverse multiple channels may be at least two different frequencies of radio waves between the radio transmitter system and radio receiver system.

It is thus another object of at least one embodiment of the invention to use frequency diversity to eliminate potential sources of interference while nevertheless ensuring that the wireless frequencies do not interfere with the MRI machine's detection of NMR signals.

The different frequencies of radio waves are transmitted alternately in time, for example, using at least one radio receiver switching between the different frequencies for reception and at least one radio transmitter switching between the different frequencies for transmission. For the radio transmitter, predetermined settle time may occur after switching and before transmitting a digital data packet.

It is thus another object of at least one embodiment of the invention to provide maximum diversity with each communicating transmitter and receiver.

The diverse multiple channels may be provided by different antennas having different polarization and or having different spatial locations.

It is thus another object of at least one embodiment of the invention to address problems unique to the shielded bore and magnet room such as create modal hot spots and drop-out zones.

The different spatial locations may be an odd multiple of one-quarter wavelength of a frequency of radio signals used to transmit the digital data packets or another distance.

It is thus another object of at least one embodiment of the invention to provide a system avoiding dead zones caused by interfering reflections off the shielded magnet room wall.

The radio receiver system may include multiple radio receivers each with switchable different antennas and wherein the receiving unit computes an error checking code or signal quality for digital data packets received on a radio receiver to selectively switch an antenna on the radio receiver when the error checking code indicates an error in. or that the signal quality comparatively low for. the digital data packet.

It is thus another object of at least one embodiment of the invention to dynamically adapt to changing conditions of the MRI room.

The diverse multiple channels may be data samples of the patient signal repeated at diverse times. For example, multiple sequential data samples of the patient signal may be collected in each digital data packet according to a rolling time window applied to the patient signal that provides for redundant data samples to be transmitted in successive digital data packets. The receiving unit may compute an error checking code or signal quality for at least two corresponding digital data packets received at different times to select one data sample of the corresponding digital data packets for outputting.

It is thus another object of at least one embodiment of the invention to create a system robust against short-duration data losses without complex and time-consuming handshaking routines.

The receiving unit may further include a radio transmitter for transmitting control instructions to the patient unit and wherein the patient unit further includes a radio receiver for receiving the control instructions from the receiving unit, for example, instructions controlling recording of data or selecting from among the patient signals or outputting an operator output display on the patient monitor.

It is yet another object of at least one embodiment of the invention to provide both data from the patient and control of the patient monitor from a remote location to the patient outside of the bore.

The radio transmitter system of the patient unit may further transmit digital data packets communicating non-patient signals via radio signals to the receiving unit.

It is thus another object of at least one embodiment of the invention to allow the patient monitor to communicate status information related to monitor hardware and operation out of the bore during monitoring when the device is not easily accessible.

The patient unit may include a battery for powering the radio transmitter system.

It is thus another object of at least one embodiment of the invention to provide diversity in a compact unit that can be contained within the bore and powered by a relatively modest battery power supply.

These particular objects and advantages may apply to only some embodiments falling within the claims and thus do not define the scope of the invention.

### BRIEF DESCRIPTION OF THE DRAWINGS

FIG. 1 is a simplified, perspective view of an MRI system showing the MRI magnet and the location of an in-bore patient unit and an out-of-bore receiving unit;

FIG. 2 is a block diagram of the patient unit of FIG. 1 configured for ECG collection and showing blocks of a microprocessor-controlled diversity transmitter employing a contained strip antenna and an on-board display;

FIG. 3 is a block diagram of the receiving unit of FIG. 1 showing multiple diversity receivers with switched antennas communicating with a programmable controller to select accurate data for outputting to a display screen;

FIG. 4 is a timing diagram of digital data packet transmitted using the diversity system of the present invention with one packet enlarged showing time diversity transmission of ECG data with a trailing error-correction code;

FIG. 5 is a figure similar to that of FIG. 4 showing a digital data packet that may be transmitted from the processing unit to the in-bore patient unit for providing commands to that transmitting unit;

FIG. 6 is a plan view of an alternative embodiment of the patient unit FIG. 2 having a graphic display;

FIG. 7 is a schematic cross-sectional representation of the graphic display employing an LED backlighting system with an LCD panel;

FIG. 8 is a perspective view of a shield container for the in-bore patient unit of FIG. 6 providing eddy-current reduction; and

FIG. 9 is a partial plan view of a patient showing a harness system for holding the patient unit of FIG. 2 to the patient in the bore for minimizing motion transmitting obstructions and lead entanglement.

### DETAILED DESCRIPTION OF THE PREFERRED EMBODIMENT

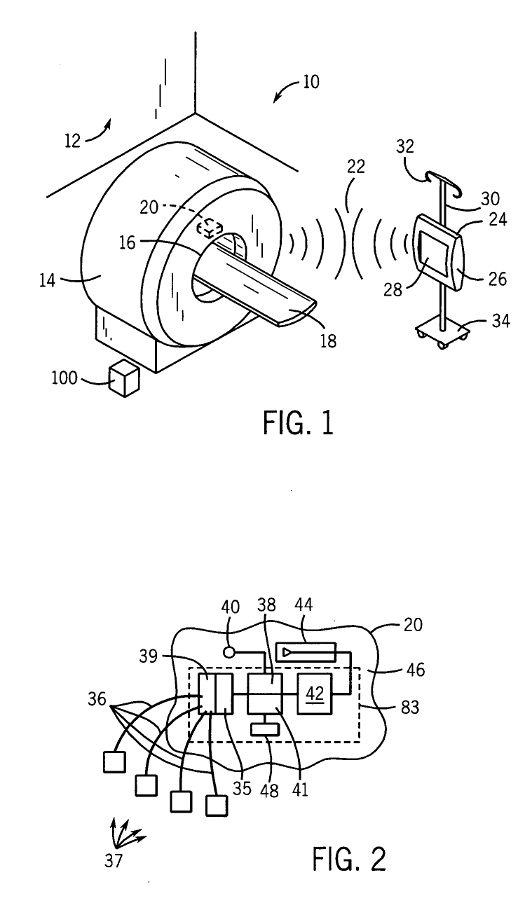

Referring now to FIG. 1, an MRI magnet room 10 containing an MRI magnet 14 may have shielded walls 12 blocking and reflecting radio waves. The MRI magnet 14 may have a central bore 16 for receiving a patient (not shown) supported on a patient table 18. As used henceforth, bore shall refer generally to the imaging volume of an MRI machine and should be considered to include the patient area between pole faces of open frame MRI systems.

During the MRI scan, the patient is held within the bore 16 and may be monitored via wireless patient unit 20 attached to the patient or patient table 18 and within the bore 16 during the scan. The patient unit 20 transmits via radio waves 22 physiological patient data and status data (as will be described) to processing unit 24 outside the bore 16 useable by personnel within the magnet room 19. The processing unit 24 typically will include controls 26 and a display 28 providing an interface for the operator, and may be usefully attached to an IV pole 30. The IV pole 30 may have hooks 32 for holding IV bags (not shown) and a rolling, weighted base 34 that may be freely positioned as appropriate without the concern for wires between the patient unit 20 and processing unit 24.

Referring now to FIG. 2, the patient unit 20 holds an interface circuit 35 for receiving physiological patient signals including, but not limited to, signals indicating: respiration, blood oxygen, blood pressure, pulse, and temperature, each from an appropriate sensor 37. Only ECG signals will be described henceforth for clarity.

When used to sense ECG signals, the interface circuit 35 may receive two or more ECG leads 36, being connected to, for example, the right arm, the right leg, the left arm and the left leg. The signals from these ECG leads 36 are connected to electrode amplifier and lead selector 39 which provides signals I, II and V, in a normal lead mode to be described below, or signals X, Y and Z in a vector lead mode (not shown), each attached to a corresponding electrode providing the sensor 37. The leads 36 may be high impedance leads so as to reduce the induction of eddy currents within those leads during the MRI process. The electrode amplifier and lead selector 39 provides the signals to an interface circuit 35 which controls signal offset and amplification, provides a gradient filter having variable filter settings to reduce interference from the MRI gradient fields, and converts the signals to digital words that may be transmitted to a contained processor 38. In a preferred embodiment, the ECG signals are sampled and digitized at a rate of 1,000 samples per second or faster so that they may be used for gating purposes. Other signals, such as those of blood oxygen may be sampled at a slower rate, for example, 250 samples per second.

The processor 38 communicates with flash memory 41 which may be used to buffer and store data from ECG leads 36 and which may have a stored program controlling the operation of the patient unit 20 as will be described below.

The processor 38 may communicate with an operator indicator 40, in this case a bi-colored LED, which may display operating information according to the following states:

| LED color | Meaning |
|---|---|
| Blinking Green | Good ECG Signals |
| Solid Green | No ECG Signal |
| Blinking Red | ECG, Poor |
| | Communication |
| Solid Red | No ECG, Poor |
| | Communication |

The operator indicator 40 has a lens which protrudes from a housing of the patient unit 20 so that it can be viewed by an operator sighting along the bore from a variety of attitudes. Importantly, the operator indicator 40 may be used during preparation of the patient outside of the bore, even in the absence of the processing unit 24 in the patient's hospital room.

The processor 38 of the patient unit 20 may also communicate with a transceiver 42. A suitable transceiver 42 provides multi-band Gaussian frequency shift keying (GFSK) in the 2.4 GHz ISM band and is capable of operating on battery power levels to produce powers of 0 dBm such as a type commercially available from Nordic Semiconductors of Norway under the trade name nRF24E1.

The transceiver 42 provides for transmission and reception of digital data packets holding samples of the ECG data with calculated error-correction codes over radio channels that may be selected by processor 38. Preferably the radio channels are selected to provide a substantial frequency difference between the channels to reduce the possibility of any interfering source of radio frequency from blocking both channels at the same time. The selection of channels 1 and 9 provide for an 8 MHz separation between channels.

The transceiver 42 connects to a microstrip antenna 44 which may be wholly contained within an insulating plastic housing 46 of the patient unit 20 outside of Faraday shield 83 to be described in more detail below. A polymer battery 48 having no ferromagnetic terminal or other components is used to provide power to each of the interface circuit 35, processor 38, transceiver 42 and operator indicator 40, all held within the Faraday shield 83.

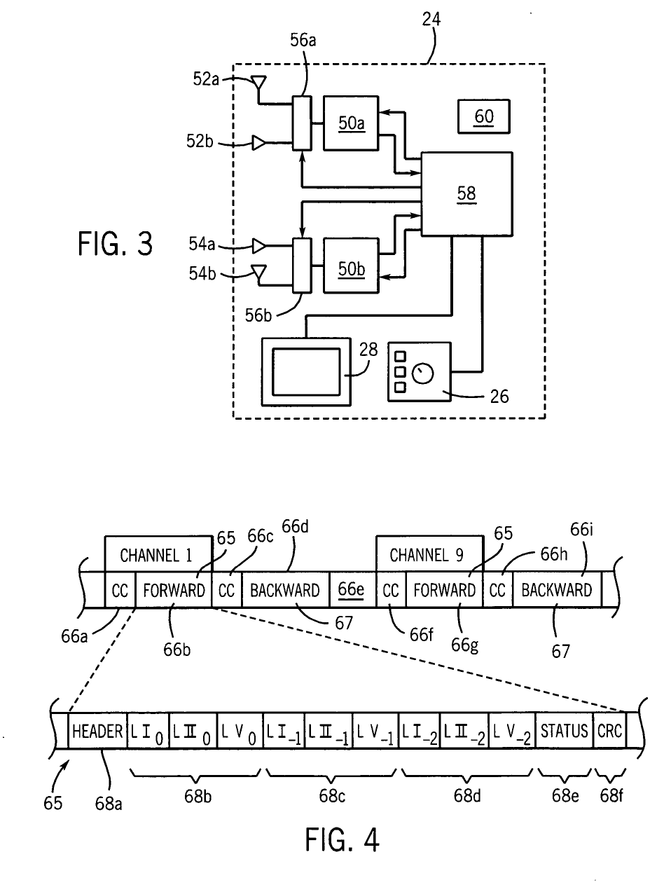

Referring now to FIG. 3, the processing unit 24 contains two transceivers 50a and 50b compatible with transceiver 42, and each switching between one of at least two channels depending on the frequency of transmission by the transceiver 42. Each of the transceivers 50 and 50b are connected to two antennas: antennas 52a and 52b for transceiver 50a, and antennas 54a and 54b for transceiver 50b, via a solid-state antenna 56a, and 56b, respectively. A controller 58 receives data from and provides data to each of transceivers 50a and 50b for communication with the patient unit 20. The controller 58 also provides signals to the 56a, and 56b to control which antennas are connected to transceiver 50a and 50b.

Antennas 52 and 54 are both spatially diverse and have different polarizations. Ideally, antennas 52a and 54a are vertically polarized and antennas 52b and 54b are horizontally polarized. Further, the antennas 52 and 54 are spaced from each other by approximately an odd multiple of a quarter wavelength of the frequencies of transmission by the patient unit 20 representing an expected separation of nodal points. This spacing will be an odd multiple of approximately 3 cm in the 2.4 GHz ISM frequency band.

With these diverse antennas 52a, 52b, 54a, and 54b, drop-off or adverse polarization of the waves at the processing unit 24, may be accommodated by switching of the antennas 52 and 54. Generally, this switching may be triggered when the signal from a given transceiver 50a or 50b is indicated to be corrupted by the error-correction code attached to data packets received by the given transceiver 50a or 50b as detected by program executed by the controller 58. Alternatively, the signal quality, for example, the signal strength or the length of time that the signal has been above a predetermined threshold, may be used to trigger the switching to the better of the two antennas 52 and 54.

The controller 58 communicates with a memory 60 such as may be used to store data and a program controlling operation of the processing unit 24. The controller 58 may also communicates with the display 28 that may display the physiological data collected by the patient unit 20 and user controls 26 that allow programming of that processing unit 24 and control of the display 28 according to methods well-known in the art.

Referring now to FIGS. 2 and 4, during operation, the processor 38 of the patient unit 20 executes a stored program in memory 60 to collect data from ECG leads 36 and to transmit it in time-diverse forward data packets 65 over multiple time frames 66. During a first time frame 66a, the processor 38 may switch the frequency of transmission of the transceiver 42 and provide a settling period of approximately 220 microseconds. As will be described, the frequency need not be changed at this time, but allowance is made for that change.

At time frame 66b, forward data packet 65, being physiological data from the patient, is transmitted from patient unit 20 to processing unit 24. This forward data packet will include a header 68 a which generally provides data needed to synchronize communication between transceivers 42 and 50a and 50b, and which identifies the particular data packet as a forward data packet 65 and identifies the type of physiological data, e.g.: ECG, SPO₂, etc.

Following the header 68 a, data 68b may be transmitted providing current samples in 16 bit digital form for the ECG signals at the current sampling time (e.g., LI₀, LII₀, LV₀). This is followed by data 68c providing corresponding samples in 16 bit digital form for the ECG signals at the next earlier sampling time (e.g., LL_{-1, LII}₋₁, LV₋₁) as buffered in the patient unit 20. This in turn is followed by data 68d providing corresponding samples in 16 bit digital form for the ECG signals at the next earlier sampling time before data 68d (e.g., LI₋₂, LII₋₂, LV₋₂) again as buffered in the patient unit 20. In the vector mode, the samples may be Xₙ, Yₙ, and Zₙ.

Thus, a rolling window of three successive sample periods (one new sample and the two previous samples for each lead) is provided for each forward data packet 65. This time diversity allows data to be transmitted even if two successive forward data packets 65 are corrupted by interference.

Status data 68e follows data 68c and provides non-physiological data from the patient unit 20 indicating generally the status of the patient unit 20 including, for the example of ECG data, measurements of lead impedance, device temperature, operating time, battery status, test information, information about the lead types selected, the gradient filter settings selected, and the next or last radio channel to be used to coordinate the transceivers 42 and 50a and 50b. The status data 68e may also include a sequence number allowing the detection of lost forward data packet 65. Different status data 68e is sent in each forward data packet 65 as indexed by all or a portion of the bits of the sequence number. This minimized the length of each forward data packets 65.

Finally status data 68e includes an error detection code 68f, for example, a cyclic redundancy code of a type well known in the art, computed over the total forward data packet 65 of header 68a, data 68b, data 68c, data 68d, and status data 68e that allows detection of corruption of the data during its transmission process by the controller 58. Detection of a corrupted forward data packet 65 using this error detection code 68f causes the controller to first see if an uncorrupted packet is available form the other transceiver 50a or 50b, and second to see if an uncorrupted packet is available from the following two forward packets. The antenna of the transceiver 50a or 50b is in any event switched to see if reception can be improved. Alternatively, signal quality, as described above, may be used to select among packets.

Referring still to FIG. 4, the forward data packet 65 of time frame 66b is followed by another channel changing time frame 66c which allows changing of the channel, if necessary, which is followed by a backward data packet 67 of time frame 66d providing data from the processing unit 24 to the patient unit 20.

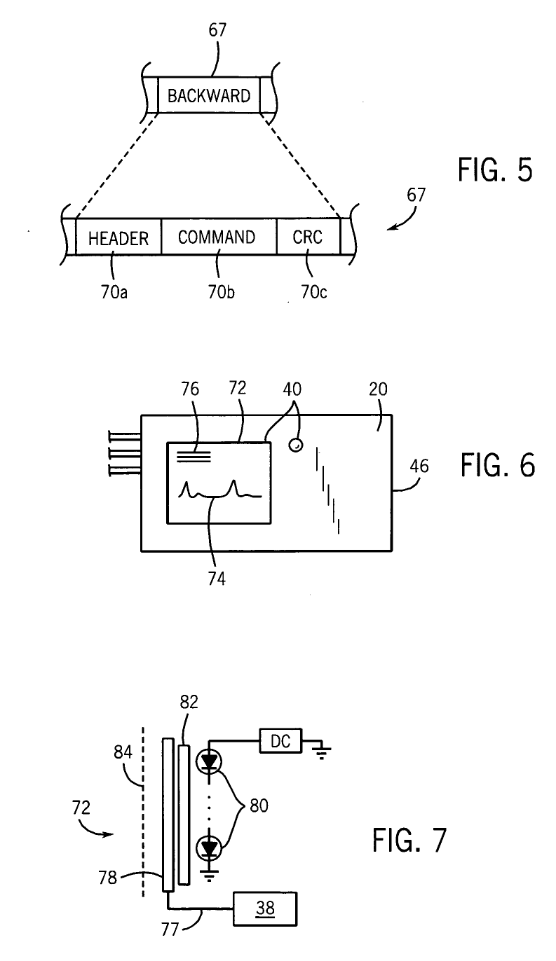

Referring now to FIG. 5, the backward data packet 67 may include a header frame 70a followed by command frame 70b and an error detection code 70c. The commands of the command frame 70b in this case may be instructions to the patient unit 20, for example, pulse the LED of the operator indicator 40 for testing or initiate a test of the hardware of the patient unit 20 according to diagnosis software contained therein, or to select the lead type of vector or normal described above, or to change the gradient filter parameters as implemented by the interface circuit 35, or to provide a calibration pulse, or to control the filling of flash memory on the patient unit 20 as may be desired.

Referring again to FIG. 4, an uncommitted time frame 66e may be provided for future use followed again by a channel change time frame 66f which typically will ensure that the radio channel used during the following forward data packet 65 of time frame 66g is different from the radio channel used in the previous forward data packet 65 of time frame 66b. This ensures frequency diversity in successive forward data packet 65 further reducing the possibility of loss of a given sample.

Referring now to FIG. 6, the present invention contemplates that the patient unit 20 may be used for setup of the patient without the need for processing unit 24, for example, in the patient's room before the patient is transported to the magnet room 10 or as a portable patient monitor that may be used for short periods of time in the patient room or during transportation of the patient and providing some of the features of the processing unit 24. For this purpose the patient unit 20 may include not only light for operator indicator 40, but graphic display 72 being similar to display 28 providing, for example, an output of physiological signal wave forms 74 and alphanumeric data 76.

Referring to FIG. 7, the display 72 to be suitable for use in the MRI environment, may comprise a liquid crystal panel 77 driven by processor 38 according to well known techniques but backlit by a series of solid state lamps, preferably white light-emitting diodes (LEDs) 80 communicating to the rear surface of the LCD panel 78 by a light pipe 82 instead of a common cold cathode fluorescent lamp. The LEDs 80 may be driven by a DC source to be unmodulated so as to reduce the possibility of creating radio frequency interference in the magnet bore caused by switching of the LEDs 80. The use of LEDs 80 also eliminates the high voltage interference that can occur from operation of cold cathode fluorescent tubes and the magnet components inherent in such tubes.

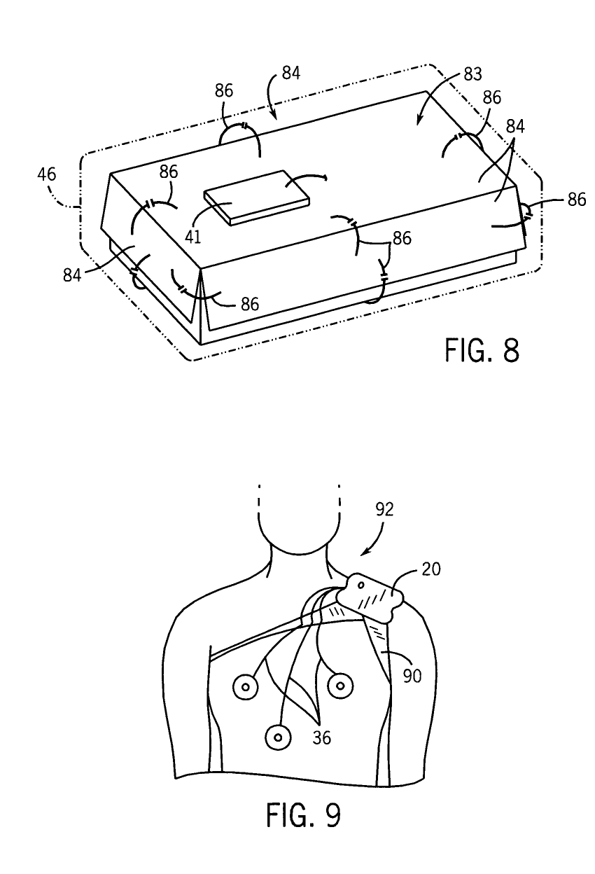

Referring now to FIG. 8, the circuitry of the patient unit 20 shown in FIG. 2, with the exception of the microstrip antenna 44, may be contained within a Faraday shield 83 held within the housing 46 and comprised of a box of conductive screen elements 84. The screen elements 84 may provide a mesh size smaller than the wavelength of the MRI gradient fields but ample to allow the display 72 to be viewed therethrough. Alternatively, the display 72 may be positioned outside of the Faraday shield 83. The light (preferably an LED) for the operator indicator 40 may protrude through the Faraday shield 83 to provide greater visibility to an operator outside the magnet bore.

The screen elements 84 providing radio frequency shielding for each face of the box forming the Faraday shield 83 may be insulated from each other with respect to direct currents, but yet joined by capacitors 86 at the corner edges of the box to allow the passage of a radio frequency current. The effect of these capacitors is to block the flow of lower frequency eddy currents induced by the magnetic gradients such as can vibrate the patient unit 20 when it is positioned on the patient.

Referring now to FIG. 9, the patient unit 20 may desirably be held by a harness 90 to the shoulder of the patient 92 so as to be free from interference with the patient while maintaining a position conducive to transmission of wireless operator indicator 40. The harness may provide a guide for the ECG leads 36 reducing their entanglement and simplifying installation of the unit on the patient 92.

Referring now to FIG. 1, the present invention further contemplates that a gating unit 100 may be positioned in the magnet room 10 to receive signals both from the processing unit 24 and patient unit 20, and thereby to generate gating signals that may be used for gating the MRI machine. This gating unit may eavesdrop on the transmissions between the patient unit 20 and the processing unit 24 reducing the transmission overhead required of using these signals for gating.

It is specifically intended that the present invention not be limited to the embodiments and illustrations contained herein, but include modified forms of those embodiments including portions of the embodiments and combinations of elements of different embodiments as come within the scope of the following claims. For example, the diversity techniques as described herein may be applicable to optical and other wireless transmission methods. In the case of optical transmission, for example, different frequencies of light, modulation types, modulation frequencies, polarizations, orientations may be used to provide diversity.

We claim:

1. A wireless patient sensor system for MRI imaging comprising;
a patient unit positionable adjacent to the patient proximate to a bore of an MRI magnet, the patient unit providing at least one sensor receiving a patient signal from the patient and having a wireless transmitter system for transmitting digital data packets communicating the patient signal;
a receiving unit having a wireless receiver system receiving the digital data packets from outside the bore of the MRI magnet for outputting information of the digital data packets; and
wherein the wireless transmitter system and wireless receiver system communicate using diverse multiple channels between the patient unit and receiving unit.

2. The wireless patient sensor system of claim 1 wherein the receiving unit computes an error checking code for the digital data packets transmitted on at least two diverse multiple channels to select one diverse multiple channel from which to obtain a digital data packet for outputting.

3. The wireless patient sensor system of claim 1 wherein the receiving unit computes an signal quality on the at least two diverse multiple channels to select one diverse multiple channel from which to obtain a digital data packet for outputting.

4. The wireless patient sensor system of claim 1 wherein the diverse multiple channels are at least two different frequencies of radio waves between the radio transmitter system and radio receiver system.

5. The wireless patient sensor system of claim 4 wherein the different frequencies of radio waves are transmitted alternately in time.

6. The wireless patient sensor system of claim 5 wherein the radio receiver system includes at least one radio receiver switching between the different frequencies for reception.

7. The wireless patient sensor system of claim 5 wherein the radio transmitter system includes at least one radio transmitter switching between the different frequencies for transmission.

8. The wireless patient sensor system of claim 7 wherein the radio transmitter waits a predetermined settle time after switching and before transmitting a digital data packet.

9. The wireless patient sensor system of claim 1 wherein the diverse multiple channels are provided by different antennas.

10. The wireless patient sensor system of claim 9 wherein the different antennas have different polarization.

11. The wireless patient sensor system of claim 9 wherein the different antennas have different spatial locations.

12. The wireless patient sensor system of claim 11 wherein the different spatial locations are an odd multiple of one-quarter wavelength of a frequency of radio signals used to transmit the digital data packets.

13. The wireless patient sensor system of claim 9 wherein the receiving unit computes an error checking code for at least two corresponding digital data packets received on different antennas to select one data packet for outputting.

14. The wireless patient sensor system of claim 9 wherein the receiving unit computes an signal quality on the at least two corresponding digital data packets received on different antennas to select one data packet for outputting.

15. The wireless patient sensor system of claim 9 wherein the diverse multiple channels are further different frequencies of radio waves between the radio transmitter system and the radio receiver system.

16. The wireless patient sensor system of claim 9 wherein the radio receiver system includes multiple radio receivers, each with switchable different antennas, and
wherein the receiving unit computes an error checking code for digital data packets received on a radio receiver to selectively switch an antenna on the radio receiver when the error checking code indicates an error in the digital data packet.

17. The wireless patient sensor system of claim 1 wherein the diverse multiple channels are data samples of the patient signal repeated at diverse times.

18. The wireless patient sensor system of claim 17 wherein multiple sequential data samples of the patient signal are collected in each digital data packet according to a rolling time window applied to the patient signal that provides for redundant data samples to be transmitted in successive digital data packets.

19. The wireless patient sensor system of claim 17 wherein the receiving unit computes an error checking code for at least two corresponding digital data packets received at different times to select one data sample of the corresponding digital data packets for outputting.

20. The wireless patient sensor system of claim 17 wherein the diverse multiple channels are further different frequencies of radio waves between the radio transmitter system and radio receiver system.

21. The wireless patient sensor system of claim 17 wherein the diverse multiple channels are further different antennas of the radio transmitter system and radio receiver systems.

22. The wireless patient sensor system of claim 1 wherein the receiving unit further includes a radio transmitter for transmitting control instructions to the patient unit, and
wherein patient unit further includes a radio receiver for receiving the control instructions from the receiving unit.

23. The wireless patient sensor system of claim 22 wherein the patient unit switchably receives multiple patient signals and includes a memory for storing patient signals and an operator output display; and
wherein the control instructions are selected from the group consisting of: start recording patient signals in memory, stop recording patient signals in memory, select from among the patient signals, output data to the operator output display.

24. The wireless patient sensor system of claim 1 wherein the radio transmitter system of the patient unit further transmits digital data packets communicating non-patient signals via radio signals to the receiving unit.

25. The wireless patient sensor system of claim 24 wherein the patient unit includes an electronic computer executing a stored program and is powered by a battery and wherein the non-patient signals are selected from the group consisting of: battery status data, patient unit temperature, a next communication channel, patient unit test information, patient unit elapsed operating time, and a patient signal processing mode.

26. The wireless patient sensor system of claim 1 wherein the sensor is an electrode, and the patient signal is ECG data.

27. The wireless patient sensor system of claim 1 wherein the sensor is an oxygen sensor, and the patient signal is blood oxygen data.

28. The wireless patient sensor system of claim 1 wherein the sensor is a respiration sensor, and the patient signal is respiration data.

29. The wireless patient sensor system of claim 1 wherein the sensor is a thermal sensor, and the patient signal is patient temperature data.

30. The wireless patient sensor system of claim 1 wherein the sensor is a blood pressure sensor, and the patient signal is blood pressure data.

31. The wireless patient sensor system of claim 1 wherein the patient unit includes a battery for powering the radio transmitter system.

32. A method of wirelessly communicating patient physiological data from a bore of an MRI magnet to a point outside the bore of the MRI magnet comprising the steps of:
(a) collecting a patient signal at a patient unit positionable adjacent to the patient proximate to the bore of an MRI magnet;
(b) transmitting digital data packets communicating the patient signal via radio signals from the patient unit;
(c) receiving the digital data packets at a receiving unit outside the bore of the MRI magnet for outputting data of the digital data packets; and
wherein the radio transmitter system and radio receiver system communicate using diverse multiple channels between the patient unit and receiving unit.

33. The method of claim 32 wherein the receiving unit computes an error checking code for the digital data packets transmitted on at least two diverse multiple channels to select one diverse multiple channel from which to obtain a digital data packet for outputting.

34. The method of claim 32 wherein the diverse multiple channels are at least two different frequencies of radio waves between the radio transmitter system and radio receiver system.

35. The method of claim 34 wherein the different frequencies of radio waves are transmitted alternately in time.

36. The method of claim 35 wherein the radio receiver system includes at least one radio receiver and including the step of switching between the different frequencies for reception at each radio receiver.

37. The method of claim 35 wherein the radio transmitter system includes at least one radio transmitter and including the step of switching between the different frequencies for transmission at each radio transmitter.

38. The method of claim 36 including the step of a predetermined settle time after switching and before transmitting a digital data packet.

39. The method of claim 32 wherein the diverse multiple channels are provided by different antennas.

40. The method of claim 39 wherein the different antennas have different polarization.

41. The method of claim 39 wherein the different antennas having different spatial locations.

42. The method of claim 41 wherein the different spatial locations are an odd multiple of one-quarter wavelength of a frequency of radio signals used to transmit the digital data packets.

43. The method of claim 39 including the step of computing an error checking code for at least two corresponding digital data packets received on different antennas to select one data packet for outputting.

44. The method of claim 39 wherein the diverse multiple channels are further different frequencies of radio waves between the radio transmitter system and the radio receiver system.

45. The method of claim 39 wherein the radio receiver system includes multiple radio receivers each with switchable different antennas and including the step of computing an error checking code for digital data packets received on a radio receiver to selectively switch an antenna on the radio receiver when the error checking code indicates an error in the digital data packet.

46. The method of claim 32 wherein the diverse multiple channels are data samples of the patient signal repeated at diverse times.

47. The method of claim 46 including the step of collecting multiple sequential data samples of the patient signal in each digital data packet according to a rolling time window applied to the patient signal that provides for redundant data samples to be transmitted in successive digital data packets.

48. The method of claim 46 including the step of computing an error checking code for at least two corresponding digital data packets received at different times to select one data sample of the corresponding digital data packets for outputting.

49. The method of claim 46 wherein the diverse multiple channels are further different frequencies of radio waves between the radio transmitter system and radio receiver system.

50. The method of claim 46 wherein the diverse multiple channels are further different antennas of the radio transmitter system and radio receiver systems.

51. The method of claim 32 wherein the receiving unit further includes a radio transmitter for transmitting control instructions to the patient unit and wherein the patient unit further includes a radio receiver for receiving the control instructions from the receiving unit and including the step of transmitting control instructions from the receiving unit to the patient unit.

52. The method of claim 51 wherein the control instructions are selected from the group consisting of: start recording patient signals in memory, stop recording patient signals in memory, select from among the patient signals, output data to an operator output display.

53. The method of claim 32 further including the step of transmitting digital data packets communicating non-patient signals via radio signals from the patient unit to the receiving unit.

54. The method of claim 53 wherein the non-patient data is selected from the group consisting of: battery status data and software revision number data.

55. The method of claim 32 wherein the patient signal is physiological data.

56. The method of claim 55 wherein the patient signal is ECG data.

57. The method of claim 55 wherein the patient signal is blood oxygen data.

58. The method of claim 55 wherein the patient signal is respiration data.

59. The method of claim 55 wherein the patient signal is patient temperature data.

60. The method of claim 55 wherein the patient signal is blood pressure data.
